(19) United States
(12) Patent Application Publication
(10) Pub. No.: US 2017/0159056 A1
(43) Pub. Date: Jun. 8, 2017

(54) ANTISENSE OLIGONUCLEOTIDES AND METHODS OF USE THEREOF

(71) Applicant: Clementia Pharmaceuticals Inc., Montreal (CA)

(72) Inventors: Eric G. MARCUSSON, San Francisco, CA (US); Donna Roy GROGAN, Boston, MA (US)

(21) Appl. No.: 15/365,376

(22) Filed: Nov. 30, 2016

Related U.S. Application Data
(60) Provisional application No. 62/378,924, filed on Aug. 24, 2016.
(60) Provisional application No. 62/261,646, filed on Dec. 1, 2015.

(51) Int. Cl. C12N 15/113

(52) U.S. Cl. CPC C12N 15/1137; C12N 2310/11; C12N 2310/32; C12N 2310/33; C12N 2310/341; C12N 2310/315; C12N 2320/10

(57) ABSTRACT

The invention is directed to antisense oligonucleotides that hybridize to the mRNA from a mutant activin A receptor type-1 (ACVR1) gene and inhibit or reduce the expression of the mutant ACVR1 gene. The mutant ACVR1 gene has the mutation c.617G>A. The invention also features pharmaceutical compositions including the antisense oligonucleotides and methods of using the antisense oligonucleotides to treat diseases or conditions (e.g., FOP and DIPG) associated with the expression of the mutant ACVR1 gene.

### BACKGROUND OF THE INVENTION

Fibrodysplasia ossificans progressiva (FOP) and diffuse intrinsic pontine glioma (DIPG) are two rare genetic diseases caused by a mutant ACVR1 gene having the mutation c.617G>A. Efforts to improve treatment and survival of subjects having these devastating diseases have not been successful. There exists a need for novel and effective treatments for FOP and DIPG.

### SUMMARY OF THE INVENTION

The present invention features antisense oligonucleotides that are targeted to a mutant activin A receptor type-1 (ACVR1) gene and inhibit or reduce the expression of the mutant ACVR1 gene that has the mutation c.617G>A. The invention also features pharmaceutical compositions including the antisense oligonucleotides and methods of using the antisense oligonucleotides to treat diseases or conditions (e.g., FOP and DIPG) associated with the expression of the mutant ACVR1 gene.

In one aspect, the invention features a single-stranded antisense oligonucleotide that is 12 to 30 nucleosides (e.g., 12, 13, 14, 15, 16, 17, 18, 19, 20, 21, 22, 23, 24, 25, 26, 27, 28, 29, or 30 nucleosides) in length, wherein the antisense oligonucleotide is complementary to an equal length portion of the sequence of TTTCTGGTACAAAGAACAGTGGCTCACCAGATTACACTGTTGGAGTGTGTC (SEQ ID NO: 1) and includes a contiguous portion that is complementary to the sequence of GCTCACCAG (SEQ ID NO: 2).

In some embodiments, the antisense oligonucleotide includes at least one modified sugar. In some embodiments, the modified sugar is selected from the group consisting of a bicyclic sugar, a 2′-O-methoxyethyl modified sugar, a 2′-methoxy modified sugar, a 2′-O-alkyl modified sugar, and an unlocked sugar. In some embodiments, the bicyclic sugar is a locked sugar.

In some embodiments, the antisense oligonucleotide includes at least one modified internucleoside linkage. In some embodiments, the modified internucleoside linkage is a phosphorothioate internucleoside linkage. In some embodiments, the antisense oligonucleotide has phosphorothioate internucleoside linkages between neighboring nucleosides throughout the length of the antisense oligonucleotide.

In some embodiments, the antisense oligonucleotide includes at least one modified nucleobase. In some embodiments, the modified nucleobase is selected from the group consisting of 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine. In some embodiments, the modified nucleobase is 5-methylcytosine.

In some embodiments of this aspect of the invention, the antisense oligonucleotide is a gapmer including a gap segment flanked by a 5′ wing segment and a 3′ wing segment.

In some embodiments, the gap segment includes 6 to 10 2′-deoxyribonucleosides (e.g., 6, 7, 8, 9, or 10 2′-deoxyribonucleosides).

In some embodiments, each of the 5′ and 3′ wing segments includes 2 to 6 nucleosides (e.g., 2, 3, 4, 5, or 6 nucleosides) and at least one modified sugar. In some embodiments, the modified sugar in each of the 5′ and 3′ wing segments is selected from the group consisting of a bicyclic sugar, a 2′-O-methoxyethyl modified sugar, a 2′-methoxy modified sugar, a 2′-O-alkyl modified sugar, and an unlocked sugar. In some embodiments, the modified sugar is a locked sugar (e.g., a locked sugar that has the 2′-oxygen linked to the 4′ ring carbon by way of a methylene). In some embodiments, each of the 5′ and 3′ wing segments includes 2 to 6 nucleosides (e.g., 2, 3, 4, 5, or 6 nucleosides) each having a locked sugar (e.g., a locked sugar that has the 2′-oxygen linked to the 4′ ring carbon by way of a methylene).

In some embodiments, the 5′ wing segment includes at least one modified nucleobase. In some embodiments, the 3′ wing segment includes at least one modified nucleobase. In some embodiments, each of the 5′ and 3′ wing segments includes at least one modified nucleobase. In some embodiments, the modified nucleobase in the 5′ and/or 3′ wing segment is 5-methylcytosine.

In some embodiments, the gapmer has phosphorothioate internucleoside linkages between neighboring nucleosides throughout the length of the gapmer.

In some embodiments, the gap segment includes 8 2′-deoxyribonucleosides, each of the 5′ and 3′ wing segments includes 4 nucleosides each having a locked sugar (e.g., a locked sugar that has the 2′-oxygen linked to the 4′ ring carbon by way of a methylene), and the gapmer has phosphorothioate internucleoside linkages between neighboring nucleosides throughout the length of the gapmer.

In some embodiments, the antisense oligonucleotide described herein is 12 to 20 nucleosides (e.g., 12, 13, 14, 15, 16, 17, 18, 19, or 20 nucleosides) in length. In some embodiments, the antisense oligonucleotide described herein is 16 nucleosides in length. In some embodiments, the antisense oligonucleotide includes the sequence of CTGGTGAGC (SEQ ID NO: 3).

In another aspect, the invention features an antisense oligonucleotide that is 16 nucleosides in length, in which each of nucleosides 1-4 and 13-16 has a locked sugar (e.g., a locked sugar that has the 2′-oxygen linked to the 4′ ring carbon by way of a methylene), each of nucleosides 5-12 is a 2′-deoxyribonucleoside, the antisense oligonucleotide has phosphorothioate internucleoside linkages between neighboring nucleosides throughout the length of the antisense oligonucleotide, and the antisense oligonucleotide is complementary to an equal length portion of the sequence of AACAGTGGCTCACCAGATTACAC (SEQ ID NO: 4) and includes a contiguous portion that is complementary to the sequence of GCTCACCAG (SEQ ID NO: 2).

In some embodiments, the antisense oligonucleotide is 16 nucleosides in length and is complementary to a sequence of any one of AACAGTGGCTCACCAG (SEQ ID NO: 5), ACAGTGGCTCACCAGA (SEQ ID NO: 6), CAGTGGCTCACCAGAT (SEQ ID NO: 7), AGTGGCTCACCAGATT (SEQ ID NO: 8), GTGGCTCACCAGATTA (SEQ ID NO: 9), TGGCTCACCAGATTAC (SEQ ID NO: 10), GGCTCACCAGATTACA (SEQ ID NO: 11), and GCTCACCAGATTACAC (SEQ ID NO: 12).

In some embodiments, the antisense oligonucleotide has a sequence of any one of CTGGTGAGCCACTGTT (SEQ ID NO: 13), TCTGGTGAGCCACTGT (SEQ ID NO: 14), ATCTGGTGAGCCACTG (SEQ ID NO: 15), AATCTGGTGAGCCACT (SEQ ID NO: 16), TAATCTGGTGAGCCAC (SEQ ID NO: 17), GTAATCTGGTGAGCCA (SEQ ID NO: 18), TGTAATCTGGTGAGCC (SEQ ID NO: 19), and GTGTAATCTGGTGAGC (SEQ ID NO: 20).

In some embodiments, the nucleobase at position 1 of the sequence of CTGGTGAGCCACTGTT (SEQ ID NO: 13) is 5-methylcytosine.

In some embodiments, the nucleobases at positions 2 and 13 of the sequence of TCTGGTGAGCCACTGT (SEQ ID NO: 14) are 5-methylcytosines.

In some embodiments, the nucleobases at positions 3 and 14 of the sequence of ATCTGGTGAGCCACTG (SEQ ID NO: 15) are 5-methylcytosines.

In some embodiments, the nucleobases at positions 4, 13, and 15 of the sequence of AATCTGGTGAGCCACT (SEQ ID NO: 16), are 5-methylcytosines.

In some embodiments, the nucleobases at positions 13, 14, and 16 of the sequence of TAATCTGGTGAGCCAC (SEQ ID NO: 17) are 5-methylcytosines.

In some embodiments, the nucleobases at positions 14 and 15 of the sequence of GTAATCTGGTGAGCCA (SEQ ID NO: 18) are 5-methylcytosines.

In some embodiments, the nucleobases at positions 15 and 16 of the sequence of TGTAATCTGGTGAGCC (SEQ ID NO: 19) are 5-methylcytosines.

In some embodiments, the nucleobase at position 16 of the sequence of GTGTAATCTGGTGAGC (SEQ ID NO: 20) is 5-methylcytosine.

In some embodiments, the antisense oligonucleotide preferentially hybridizes to a mutant activin A receptor type-1 (ACVR1) gene over a wild-type ACVR1 gene, wherein the mutant ACVR1 gene has the mutation c.617G>A.

In another aspect, the invention features a pharmaceutical composition including any one of the antisense oligonucleotides described herein and one or more pharmaceutically acceptable carriers or excipients.

In another aspect, the invention features a method of inhibiting the expression of a mutant ACVR1 gene in a subject. The method includes administering to the subject a therapeutically effective amount of an antisense oligonucleotide or a pharmaceutical composition described herein, wherein the mutant ACVR1 gene has the mutation c.617G>A.

In another aspect, the invention features a method of treating a subject having a disease or condition associated with the expression of a mutant activin A receptor type-1 (ACVR1) gene. The method includes administering to the subject a therapeutically effective amount of an antisense oligonucleotide or a pharmaceutical composition described herein, wherein the mutant ACVR1 gene has the mutation c.617G>A and wherein the antisense oligonucleotide inhibits the expression of the mutant ACVR1 gene.

In some embodiments of this aspect, the disease is FOP.

In some embodiments of this aspect, the disease is DIPG.

In another aspect, the invention features a method of preventing or reducing heterotopic ossification in a subject who has FOP. The method includes administering to the subject a therapeutically effective amount of an antisense oligonucleotide or a pharmaceutical composition described herein, wherein the subject has a mutant ACVR1 gene, wherein the mutant ACVR1 gene has the mutation c.617G>A, and wherein the antisense oligonucleotide inhibits the expression of the mutant ACVR1 gene.

In some embodiments of the methods of invention described herein, the antisense oligonucleotide preferentially hybridizes to a mutant ACVR1 gene over a wild-type ACVR1 gene.

### DEFINITIONS

As used herein, the term “antisense oligonucleotide” refers to an oligomer or polymer of nucleosides, such as naturally-occurring nucleosides (i.e., adenosine, guanosine, cytidine, 5-methyluridine, or uridine) or modified forms thereof, that are covalently linked to each other though internucleoside linkages. An antisense oligonucleotide is complementary to a target nucleic acid, such that the antisense oligonucleotide hybridizes to the target nucleic acid sequence. A modified form of a nucleoside, or a modified nucleoside, refers to a nucleoside that has at least one change that is structurally distinguishable from a naturally-occurring nucleoside. In some embodiments, a modified nucleoside includes a modified nucleobase and/or a modified sugar.

As used herein, the term “hybridize” or “hybridization” refers to the annealing of complementary nucleic acids (i.e., an antisense oligonucleotide and its target nucleic acid) through hydrogen bonding interactions that occur between complementary nucleobases, nucleosides, or nucleotides. The hydrogen bonding interactions may be Watson-Crick hydrogen bonding or Hoogsteen or reverse Hoogsteen hydrogen bonding. Examples of complementary nucleobase pairs include, but are not limited to, adenine and thymine, cytosine and guanine, and adenine and uracil, which all pair through the formation of hydrogen bonds.

As used herein, the term “complementary” refers to the capacity for precise pairing between nucleobases, nucleosides, or nucleotides. For example, if a nucleoside at a certain position of an antisense oligonucleotide is capable of hydrogen bonding with a nucleoside at the same position of the target nucleic acid sequence of the antisense oligonucleotide, then the antisense oligonucleotide and its target nucleic acid sequence are considered to be complementary at that position.

As used herein, the term “nucleobase” refers to a heterocyclic base moiety capable of forming hydrogen bonds with another nucleobase. Nucleobases provide the hydrogen bonding interactions that are needed bind or hybridize one nucleic acid strand to another in a sequence specific manner. A nucleobase may be a naturally occurring nucleobase (i.e., adenine, guanine, cytosine, thymine, or uracil) or a modified nucleobase. Examples of modified nucleobases are described in detail further herein.

As used herein, the term “nucleoside” refers to a nucleobase linked to a sugar (i.e., a pentofuranosyl sugar). A nucleoside may be a naturally occurring nucleoside (i.e., adenosine, guanosine, cytidine, 5-methyluridine, or uridine) or a modified nucleoside. A modified nucleoside includes a modified nucleobase and/or a modified sugar. Examples of modified nucleobases and modified sugars are described in detail further herein.

As used herein, the term “nucleotide” refers a nucleobase covalently linked to a sugar and a 5′ functional moiety (e.g., a phosphorous moiety). In other words, a nucleotide includes a nucleoside and a 5′ functional moiety (e.g., a phosphorous moiety) covalently linked to the 5′ carbon of the sugar portion of the nucleoside. A 5′ functional moiety in a nucleotide refers to a functional group that is covalently attached to the 5′ carbon of the sugar and generally serves to connect neighboring nucleotides (i.e., the functional moiety joined to the 5′ carbon of the sugar of one nucleoside is covalently linked to the 3′ carbon of the sugar of the adjacent nucleoside). An example of a 5′ functional moiety is a phosphorous moiety, which refers to a phosphorous-containing functional moiety that is covalently linked to the 5′ carbon of the sugar and functions to connect neighboring nucleotides. Examples of phosphorous moieties include, but are not limited to, a phosphate, a phosphorothioate, a phosphorodithioate, a phosphoramidate, a phosphorodiamidate, a thiophosphoramidate, and a thiophosphorodiamidate. The 5′ functional moiety (e.g., a phosphorous moiety) of a nucleotide forms part of the internucleoside linkage, which is defined further herein.

A nucleotide may be a naturally-occurring nucleotide or a modified nucleotide. A naturally-occurring nucleotide has a naturally-occurring nucleoside (i.e., adenosine, guanosine, cytidine, 5-methyluridine, or uridine) covalently linked to a phosphate at the 5′ carbon of the sugar. A modified nucleotide refers to a nucleotide having at least one change that is structurally distinguishable from a naturally-occurring nucleotide. A modified nucleotide may include a modified nucleobase and/or a modified sugar. Examples of modified nucleobases and modified sugars are described in detail further herein.

As used herein, the term “modified nucleobase” refers to a nucleobase having at least one change from a naturally-occurring nucleobase (i.e., adenine, guanine, cytosine, thymine, or uracil).

As used herein, the term “modified sugar” refers to a sugar having at least one change from a naturally-occurring sugar (i.e., 2′-deoxyribose in DNA or ribose in RNA). In some embodiments, a modified sugar is a pentofuranosyl sugar. In some embodiments, a modified sugar is a locked sugar. In some embodiments, a modified sugar is an unlocked sugar.

As used here, the term “internucleoside linkage” refers to the backbone linkage of the oligonucleotide that connects the neighboring nucleosides. An internucleoside linkage may be a naturally-occurring internucleoside linkage (i.e., a phosphate linkage, also referred to as a 3′ to 5′ phosphodiester linkage) or a modified internucleoside linkage. As used herein, the term “modified internucleoside linkage” refers to an internucleoside linkage having at least one change from a naturally-occurring internucleoside linkage. Examples of modified internucleoside linkages include, but are not limited to, a phosphorothioate linkage, a phosphorodithioate linkage, a phosphoramidate linkage, a phosphorodiamidate linkage, a thiophosphoramidate linkage, a thiophosphorodiamidate linkage, a phosphoramidate morpholino linkage, and a thiophosphoramidate morpholino linkage, and a thiophosphorodiamidate morpholino linkage, which are known in the art and described in, e.g., Bennett and Swayze, Annu Rev Pharmacol Toxicol. 50:259-293, 2010.

As used herein, the term “phosphorothioate linkage” refers to a 3′ to 5′ phosphodiester linkage that has a sulfur atom for a non-bridging oxygen in the phosphate backbone of an oligonucleotide.

As used herein, the term “phosphorodithioate linkage” refers to a 3′ to 5′ phosphodiester linkage that has two sulfur atoms for non-bridging oxygens in the phosphate backbone of an oligonucleotide.

As used herein, the term “thiophosphoramidate linkage” refers to a 3′ to 5′ phospho-linkage that has a sulfur atom for a non-bridging oxygen and a NH group as the 3′-bridging oxygen in the phosphate backbone of an oligonucleotide.

As used herein, the term “bicyclic sugar” refers to a modified pentofuranosyl sugar containing two fused rings. For example, a bicyclic sugar may have the 2′ ring carbon of the pentofuranose linked to the 4′ ring carbon by way of one or more carbons (i.e., a methylene) and/or heteroatoms (i.e., sulfur, oxygen, or nitrogen). An example of a bicyclic sugar is a locked sugar.

As used herein, the term “locked sugar” refers to a pentofuranosyl sugar in which the 2′-oxygen is linked to the 4′ ring carbon by way of a carbon (i.e., a methylene) or a heteroatom (i.e., sulfur, oxygen, or nitrogen). In some embodiments, a locked sugar has the 2′-oxygen linked to the 4′ ring carbon by way of a carbon (i.e., a methylene). A nucleoside having a locked sugar is referred to as a locked nucleoside.

As used herein, the term “unlocked sugar” refers to an acyclic sugar that has a 2′, 3′-seco acyclic structure, where the bond between the 2′ carbon and the 3′ carbon in a pentofuranosyl ring is absent.

As used herein, the term “gapmer” refers to a type of antisense oligonucleotide that includes a gap segment flanked by a 5′ wing segment and a 3′ wing segment. The gap segment generally serves to target the region of the nucleic acid that is hybridized to the antisense oligonucleotide for endonuclease cleavage. In some embodiments, the gap segment includes 6-10 2′-deoxyribunucleosides. The 5′ wing segment flanks the 5′ terminus of the gap segment and similarly, the 3′ wing segment flanks the 3′ terminus of the gap segment. The gap segment and the wing segments are chemically distinct. Each of the 5′ and 3′ wing segments includes at least one modified nucleoside. The modified nucleoside in each of the 5′ and 3′ wing segments may include a modified nucleobase and/or a modified sugar. In some embodiments, the modified nucleoside includes a locked sugar. The 5′ and 3′ wing segments function to protect the internal gap segment from nuclease degradation. In some embodiments, each of the 5′ and 3′ wing segments has 2-6 nucleosides. In some embodiments, a gapmer includes one or more modified internucleoside linkages. The modified internucleoside linkages may be in the gap segment and/or the 5′ and 3′ wing segments.

As used herein, the term “c.617G>A” refers to the mutation found in FOP and DIPG in which the nucleotide at cDNA position 617 of the ACVR1 gene is mutated from G (nucleotide guanine found in the wild-type ACVR1 gene) to A (nucleotide adenine found in the mutant ACVR1 gene).

### DETAILED DESCRIPTION OF THE INVENTION

The present invention employs antisense oligonucleotides for use in inhibiting or reducing the expression of a mutant activin A receptor type-1 (ACVR1) gene. The present invention describes antisense oligonucleotides that target and bind to a mutant ACVR1 gene, pharmaceutical compositions including the antisense oligonucleotides, and methods of treating diseases and conditions, e.g., fibrodysplasia ossificans progressiva (FOP) or diffuse intrinsic pontine glioma (DIPG), associated with the expression of a mutant ACVR1 gene using the antisense oligonucleotides. The mutant ACVR1 gene has the mutation c.617G>A. The sequence of a human, wild-type ACVR1 gene is shown in NCBI Gene ID NO: 90.

### I. Antisense Oligonucleotides

An antisense oligonucleotides described herein is an oligomer or polymer of nucleosides that target and bind specifically to a mutant AVCR1 gene having the mutation c.617G>A. The antisense oligonucleotide is complementary to a region of the mutant ACVR1 gene, such that the antisense oligonucleotide hybridizes to the mutant ACVR1 gene. In some embodiments, the antisense oligonucleotide hybridizes to the mutant AVCR1 gene and activates endonuclease cleavage, i.e., RNaseH cleavage, of the mutant AVCR1 gene. In some embodiments, the antisense oligonucleotide preferentially hybridizes to the mutant ACVR1 gene having the mutation c.617G>A over a wild-type ACVR1 gene. In some embodiments, the antisense oligonucleotide preferentially hybridizes to the mutant ACVR1 gene if it hybridizes to the mutant ACVR1 gene at least 20% more (i.e., at least 20%, 30%, 40%, 50%, 60%, 70%, 80%, or 90% more, twice more, or three times more, etc.) than it hybridizes a wild-type human ACVR1 gene under identical conditions.

The antisense oligonucleotide is complementary to an equal length portion of the sequence of TTTCTGGTACAAAGAACAGTGGCTCACCAGATTACACTGTTGGAGTGTGTC (SEQ ID NO: 1). The antisense oligonucleotide also includes a contiguous portion that is complementary to the sequence of GCTCACCAG (SEQ ID NO: 2). In some embodiments, an antisense oligonucleotide includes 12 to 30 nucleosides (e.g., 12, 13, 14, 15, 16, 17, 18, 19, 20, 21, 22, 23, 24, 25, 26, 27, 28, 29, or 30 nucleosides (e.g., 16 nucleosides)). In some embodiments, an antisense oligonucleotide includes 12 to 20 nucleosides (e.g., 12, 13, 14, 15, 16, 17, 18, 19, or 20 nucleosides (e.g., 16 nucleosides)). In some embodiments, an antisense oligonucleotide includes 16 nucleosides, in which each of nucleosides 1-4 and 13-16 has a modified sugar (e.g., a locked sugar), each of nucleosides 5-12 is a 2′-deoxyribonucleoside, and the antisense oligonucleotide is complementary to an equal length portion of the sequence of AACAGTGGCTCACCAGATTACAC (SEQ ID NO: 4) and includes a contiguous portion that is complementary to the sequence of GCTCACCAG (SEQ ID NO: 2). In some embodiments, an antisense oligonucleotide includes 16 nucleosides and is complementary to the sequence of any one of AACAGTGGCTCACCAG (SEQ ID NO: 5), ACAGTGGCTCACCAGA (SEQ ID NO: 6), CAGTGGCTCACCAGAT (SEQ ID NO: 7), AGTGGCTCACCAGATT (SEQ ID NO: 8), GTGGCTCACCAGATTA (SEQ ID NO: 9), TGGCTCACCAGATTAC (SEQ ID NO: 10), GGCTCACCAGATTACA (SEQ ID NO: 11), and GCTCACCAGATTACAC (SEQ ID NO: 12). In some embodiments, an antisense oligonucleotide has the sequence of any one of CTGGTGAGCCACTGTT (SEQ ID NO: 13), TCTGGTGAGCCACTGT (SEQ ID NO: 14), ATCTGGTGAGCCACTG (SEQ ID NO: 15), AATCTGGTGAGCCACT (SEQ ID NO: 16), TAATCTGGTGAGCCAC (SEQ ID NO: 17), GTAATCTGGTGAGCCA (SEQ ID NO: 18), TGTAATCTGGTGAGCC (SEQ ID NO: 19), and GTGTAATCTGGTGAGC (SEQ ID NO: 20).

In any one of the antisense oligonucleotides described above, the antisense oligonucleotide may include at least one modified nucleoside. In some embodiments, the antisense oligonucleotide includes at least one modified nucleobase (e.g., 5-methylcytosine), at least one modified sugar (e.g., a locked sugar (i.e., a locked sugar that has the 2′-oxygen linked to the 4′ ring carbon by way of a methylene)), and/or at least one modified internucleoside linkage (e.g., a phosphorothioate linkage). In some embodiments, an antisense oligonucleotide described herein has at least one phosphorothioate linkage. In some embodiments, all of the internucleoside linkages in an antisense oligonucleotide described herein are phosphorothioate linkages. In some embodiments, an antisense oligonucleotide that binds to a mutant AVCR1 gene is a gapmer, which activates endonuclease cleavage, i.e., RNaseH cleavage, of the mutant AVCR1 gene.

In some embodiments, antisense oligonucleotides described herein may be covalently linked to one or more moieties or conjugates which enhance the activity, cellular distribution, and/or cellular uptake of the resulting antisense oligonucleotides. Typical conjugate groups include, but are not limited to, cholesterol moieties and lipid moieties. Other conjugate groups include, but are not limited to, carbohydrates, phospholipids, peptides, antibodies, biotin, phenazine, folate, phenanthridine, anthraquinone, acridine, fluoresceins, rhodamines, coumarins, dyes, and other small molecules. In some embodiments, antisense oligonucleotides described herein may also be modified to have one or more stabilizing groups that are generally attached to one or both termini of antisense oligonucleotides to enhance properties such as, for example, nuclease stability. Stabilizing groups include, e.g., cap structures. These terminal modifications protect the antisense oligonucleotide having terminal nucleic acid from exonuclease degradation, and can help in delivery and/or localization of the antisense oligonucleotide within a cell. The cap can be present at the 5′-terminus (5′-cap), or at the 3′-terminus (3′-cap), or can be present on both termini. Cap structures are well-known in the art and include, for example, inverted deoxy abasic caps.

### II. Gapmer

An antisense oligonucleotide described herein that hybridizes to a mutant ACVR1 gene may be a gapmer, which is a type of antisense oligonucleotide. A gapmer includes a gap segment flanked by a 5′ wing segment and a 3′ wing segment. The gap segment generally serves to target the mutant AVCR1 gene for endonuclease cleavage, i.e., RNaseH cleavage. The 5′ and 3′ wing segments function to protect the internal gap segment from nuclease degradation and to increase the binding affinity for the target mRNA. In some embodiments, various gapmers are designed such that the complement of the mutation c.6178G>A in the mutant AVCR1 gene appears at each position of the gap segment in the gapmer. In some embodiments, the gap segment includes 2′-deoxyribunucleosides (i.e., 6-10 2′-deoxyribunucleosides). In some embodiments, the gap segment includes at least one modified nucleoside. The 5′ wing segment flanks the 5′ terminus of the gap segment and similarly, the 3′ wing segment flanks the 3′ terminus of the gap segment. A gapmer described herein includes a contiguous portion that is complementary to the sequence of GCTCACCAG (SEQ ID NO: 2).

In some embodiments, each of the 5′ and 3′ wing segments includes at least one modified nucleoside. In some embodiments, the modified nucleoside in each of the 5′ and 3′ wing segments may include a modified nucleobase (e.g., 5-methylcytosine) and/or a modified sugar (e.g., a locked sugar). The gap segment and the wing segments are chemically distinct. In some embodiments, the regions of a gapmer are differentiated by the types of sugars (i.e., naturally-occurring sugars or modified sugars) in each distinct region. In some embodiments, the gap segment may include 2′-deoxyribunucleosides, while the 5′ and 3′ wing segments include nucleosides having a locked sugar. In some embodiments, each of the 5′ and 3′ wing segments has 2-6 nucleosides. In some embodiments, a gapmer described herein includes at least one modified internucleoside linkage (e.g., a phosphorothioate linkage). In some embodiments, all of the internucleoside linkages in a gapmer described herein are phosphorothioate linkages.

In general, the wing-gap-wing motif in a gapmer is frequently described as “X-Y-Z,” in which “X” represents the length of the 5′ wing segment, “Y” represents the length of the gap segment, and “Z” represents the length of the 3′ wing segment. In some embodiments, each of X and Z is 2-6 nucleosides in length (e.g., 2, 3, 4, 5, or 6 nucleosides long). In some embodiments, Y is 6-10 nucleosides long (e.g., 6, 7, 8, 9, or 10 nucleosides in length). In some embodiments, an antisense oligonucleotide described herein is a 4-8-4 gapmer. In some embodiments, an antisense oligonucleotide is a 4-8-4 gapmer having 16 nucleosides and is complementary to the sequence of AACAGTGGCTCACCAG (SEQ ID NO: 5), ACAGTGGCTCACCAGA (SEQ ID NO: 6), CAGTGGCTCACCAGAT (SEQ ID NO: 7), AGTGGCTCACCAGATT (SEQ ID NO: 8), GTGGCTCACCAGATTA (SEQ ID NO: 9), TGGCTCACCAGATTAC (SEQ ID NO: 10), GGCTCACCAGATTACA (SEQ ID NO: 11), or GCTCACCAGATTACAC (SEQ ID NO: 12). In some embodiments, an antisense oligonucleotide is a 4-8-4 gapmer having the sequence of CTGGTGAGCCACTGTT (SEQ ID NO: 13), TCTGGTGAGCCACTGT (SEQ ID NO: 14), ATCTGGTGAGCCACTG (SEQ ID NO: 15), AATCTGGTGAGCCACT (SEQ ID NO: 16), TAATCTGGTGAGCCAC (SEQ ID NO: 17), GTAATCTGGTGAGCCA (SEQ ID NO: 18), TGTAATCTGGTGAGCC (SEQ ID NO: 19), or GTGTAATCTGGTGAGC (SEQ ID NO: 20).

In some embodiments, in any one of the 4-8-4 gapmers described above (e.g., gapmers having sequences of SEQ ID NOs: 13-20), each of nucleosides 1-4 and 13-16 has a modified sugar (e.g., a locked sugar (i.e., a locked sugar that has the 2′-oxygen linked to the 4′ ring carbon by way of a methylene)), each of nucleosides 5-12 is a 2′-deoxyribonucleoside, and all of the internucleoside linkages in the gapmer are phosphorothioate linkages. In some embodiments, each of the 5′ and 3′ wing segments in a gapmer (e.g., a gapmer having a sequence of any one of SEQ ID NOs: 13-20) has one or more modified nucleobases (e.g., 5-methylcytosine). In some embodiments, the nucleobase at position 1 of the sequence of CTGGTGAGCCACTGTT (SEQ ID NO: 13) is 5-methylcytosine. In some embodiments, the nucleobases at positions 2 and 13 of the sequence of TCTGGTGAGCCACTGT (SEQ ID NO: 14) are 5-methylcytosines. In some embodiments, the nucleobases at positions 3 and 14 of the sequence of ATCTGGTGAGCCACTG (SEQ ID NO: 15) are 5-methylcytosines. In some embodiments, the nucleobases at positions 4, 13, and 15 of the sequence of AATCTGGTGAGCCACT (SEQ ID NO: 16), are 5-methylcytosines. In some embodiments, the nucleobases at positions 13, 14, and 16 of the sequence of TAATCTGGTGAGCCAC (SEQ ID NO: 17) are 5-methylcytosines. In some embodiments, the nucleobases at positions 14 and 15 of the sequence of GTAATCTGGTGAGCCA (SEQ ID NO: 18) are 5-methylcytosines. In some embodiments, the nucleobases at positions 15 and 16 of the sequence of TGTAATCTGGTGAGCC (SEQ ID NO: 19) are 5-methylcytosines. In some embodiments, the nucleobase at position 16 of the sequence of GTGTAATCTGGTGAGC (SEQ ID NO: 20) is 5-methylcytosine.

### III. Modified Nucleobases

A modified nucleobase (or base) refers to a nucleobase having at least one change that is structurally distinguishable from a naturally-occurring nucleobase (i.e., adenine, guanine, cytosine, thymine, or uracil). In some embodiments, a modified nucleobase is functionally interchangeable with its naturally-occurring counterpart. Both naturally-occurring and modified nucleobases are capable of hydrogen bonding. Modifications on modified nucleobases may help to improve the stability of the antisense oligonucleotides to nucleases, increase binding affinity of the antisense oligonucleotides to their target nucleic acids, and decrease off-target binding of the antisense oligonucleotides. In some embodiments, an antisense oligonucleotide described herein may include at least one modified nucleobase. Examples of modified nucleobases include, but are not limited to, 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine. In some embodiments, an antisense oligonucleotide described herein has one or more modified nucleobases (e.g., 5-methylcytosine). In some embodiments, a gapmer described herein (e.g., a gapmer having a sequence of any one of SEQ ID NOs: 13-20) has one or more modified nucleobases (e.g., 5-methylcytosine) in the 5′ wing segment and/or the 3′ wing segment.

### IV. Modified Sugars

A modified sugar refers to a sugar having at least one change that is structurally distinguishable from a naturally-occurring sugar (i.e., 2′-deoxyribose in DNA or ribose in RNA). Modifications on modified sugars may help to improve the stability of the antisense oligonucleotides to nucleases, increase binding affinity of the antisense oligonucleotides to their target nucleic acids, and decrease off-target binding of the antisense oligonucleotides. In some embodiments, the sugar is a pentofuranosyl sugar. The pentofuranosyl sugar ring of a nucleoside may be modified in various ways including, but not limited to, addition of a substituent group, particularly, at the 2′ position of the ring; bridging two non-geminal ring atoms to form a bicyclic sugar (i.e., a locked sugar); and substitution of an atom or group such as —S—, —N(R)— or —C(R₁)(R₂) for the ring oxygen. Examples of modified sugars include, but are not limited to, substituted sugars, especially 2′-substituted sugars having a 2′-F, 2′-OCH2 (2′-OMe), or a 2′-O(CH₂)₂—OCH3(2′-O-methoxyethyl or 2′-MOE) substituent group; and bicyclic sugars. A bicyclic sugar refers to a modified pentofuranosyl sugar containing two fused rings. For example, a bicyclic sugar may have the 2′ ring carbon of the pentofuranose linked to the 4′ ring carbon by way of one or more carbons (i.e., a methylene) and/or heteroatoms (i.e., sulfur, oxygen, or nitrogen). The second ring in the sugar limits the flexibility of the sugar ring and thus, constrains the oligonucleotide in a conformation that is favorable for base pairing interactions with its target nucleic acids. An example of a bicyclic sugar is a locked sugar, which is a pentofuranosyl sugar having the 2′-oxygen linked to the 4′ ring carbon by way of a carbon (i.e., a methylene) or a heteroatom (i.e., sulfur, oxygen, or nitrogen). In some embodiments, a locked sugar has the 2′-oxygen linked to the 4′ ring carbon by way of a carbon (i.e., a methylene). In other words, a locked sugar has a 4′-(CH₂)—O-2′ bridge, such as α-L-methyleneoxy (4′-CH₂—O-2′) and β-D-methyleneoxy (4′-CH₂—O-2′). A nucleoside having a lock sugar is referred to as a locked nucleoside.

Other examples of bicyclic sugars include, but are not limited to, (6'S)-6′ methyl bicyclic sugar, aminooxy (4′-CH₂—O—N(R)-2′) bicyclic sugar, oxyamino (4′-CH₂—N(R)—O-2′) bicyclic sugar, wherein R is, independently, H, a protecting group or C1-C12 alkyl. The substituent at the 2′ position can also be selected from allyl, amino, azido, thio, O-allyl, O—C1-C10 alkyl, OCF₃, O(CH₂)₂SCH₃, O(CH₂)₂—O—N(Rₘ)(Rₙ), and O—CH₂—C(═O)—N(Rₘ)(Rₙ), wherein each Rₘ and Rₙ is, independently, H or substituted or unsubstituted C1-C10 alkyl.

In some embodiments, a modified sugar is an unlocked sugar. An unlocked sugar refers to an acyclic sugar that has a 2′, 3′-seco acyclic structure, where the bond between the 2′ carbon and the 3′ carbon in a pentofuranosyl ring is absent.

In some embodiments, an antisense oligonucleotide of the invention is a gapmer (e.g., a 4-8-4 gapmer), in which each of the nucleosides in the 5′ and 3′ wing segments of the gapmer has a modified sugar (e.g., a locked sugar (i.e., a locked sugar that has the 2′-oxygen linked to the 4′ ring carbon by way of a methylene)) and each of the nucleosides in the gap segment has a 2′-deoxyribose.

### V. Modified Internucleoside Linkages

An internucleoside linkage refers to the backbone linkage that connects the nucleosides. An internucleoside linkage may be a naturally-occurring internucleoside linkage (i.e., a phosphate linkage, also referred to as a 3′ to 5′ phosphodiester linkage, which is found in DNA and RNA) or a modified internucleoside linkage. A modified internucleoside linkage refers to an internucleoside linkage having at least one change that is structurally distinguishable from a naturally-occurring internucleoside linkage. Modified internucleoside linkages may help to improve the stability of the antisense oligonucleotides to nucleases and enhance cellular uptake.

Examples of modified internucleoside linkages include, but are not limited to, a phosphorothioate linkage, a phosphorodithioate linkage, a phosphoramidate linkage, a phosphorodiamidate linkage, a thiophosphoramidate linkage, a thiophosphorodiamidate linkage, a phosphoramidate morpholino linkage, and a thiophosphoramidate morpholino linkage, and a thiophosphorodiamidate morpholino linkage, which are known in the art and described in, e.g., Bennett and Swayze, Annu Rev Pharmacol Toxicol. 50:259-293, 2010. A phosphorothioate linkage is a 3′ to 5′ phosphodiester linkage that has a sulfur atom for a non-bridging oxygen in the phosphate backbone of an oligonucleotide. A phosphorodithioate linkage is a 3′ to 5′ phosphodiester linkage that has two sulfur atoms for non-bridging oxygens in the phosphate backbone of an oligonucleotide. A thiophosphoramidate linkage refers to a 3′ to 5′ phospho-linkage that has a sulfur atom for a non-bridging oxygen and a NH group as the 3′-bridging oxygen in the phosphate backbone of an oligonucleotide. In some embodiments, an antisense oligonucleotide described herein has at least one phosphorothioate linkage. In some embodiments, all of the internucleoside linkages in an antisense oligonucleotide described herein are phosphorothioate linkages.

### VI. Pharmaceutical Compositions and Preparations

The invention features pharmaceutical compositions that include an antisense oligonucleotide described herein. In addition to the antisense oligonucleotide, the pharmaceutical compositions may contain one or more pharmaceutically acceptable carriers or excipients, which can be formulated by methods known to those skilled in the art. In some embodiments, a pharmaceutical composition of the present invention includes an antisense oligonucleotide in a therapeutically effective amount. In certain embodiments, the therapeutically effective amount of the antisense oligonucleotide is sufficient to prevent, alleviate, or ameliorate symptoms of a disease or to prolong the survival of the subject being treated. Determination of a therapeutically effective amount is within the capability of those skilled in the art.

Antisense oligonucleotides may be mixed with pharmaceutically acceptable active and/or inert substances for the preparation of pharmaceutical compositions. Compositions and methods for the formulation of pharmaceutical compositions are dependent upon a number of criteria, including, but not limited to, route of administration, extent of disease, or dose to be administered. An antisense oligonucleotide targeted to a mutant ACVR1 gene having the mutation c.617G>A can be utilized in pharmaceutical compositions by combining the antisense oligonucleotide with a suitable pharmaceutically acceptable diluent or carrier. A pharmaceutically acceptable diluent includes phosphate-buffered saline (PBS). PBS is a diluent suitable for use in compositions to be delivered parenterally. In some embodiments, a pharmaceutical composition includes an antisense oligonucleotide described herein and a pharmaceutically acceptable diluent. In some embodiments, the pharmaceutically acceptable diluent is PBS.

Pharmaceutical compositions including antisense oligonucleotides encompass any pharmaceutically acceptable salts or esters thereof, which, upon administration to a mammal (i.e., a human), is capable of providing (directly or indirectly) the biologically active form of the antisense oligonucleotide. Accordingly, for example, the disclosure is also drawn to pharmaceutically acceptable salts of antisense oligonucleotides, prodrugs, pharmaceutically acceptable salts of such prodrugs, and other bioequivalents. Suitable pharmaceutically acceptable salts include, but are not limited to, sodium and potassium salts. In some embodiments, a prodrug can include the incorporation of additional nucleosides or nucleotides at one or both ends of an antisense oligonucleotide which are cleaved by endogenous nucleases within the body, to form the active antisense oligonucleotide.

In some embodiments, pharmaceutical compositions of the present invention include one or more oligonucleotides and one or more pharmaceutically acceptable carriers or excipients. Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. In some embodiments, carriers and excipients are selected from water, salt solutions, alcohol, polyethylene glycols, gelatin, lactose, amylase, magnesium stearate, talc, silicic acid, viscous paraffin, hydroxymethylcellulosem, and polyvinylpyrrolidone. In some embodiments, a pharmaceutical composition of the present invention includes a co-solvent system. Examples of co-solvent systems include, but are not limited to, benzyl alcohol, a nonpolar surfactant, a water-miscible organic polymer, and an aqueous phase. In some embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™, and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In some embodiments, a pharmaceutical composition of the present invention is prepared using known techniques, including, but not limited to mixing, dissolving, granulating, dragee-making, levigating, emulsifying, encapsulating, entrapping, and tabletting processes. In some embodiments, a pharmaceutical composition of the present invention is a liquid (e.g., a suspension, elixir and/or solution). In some embodiments, a liquid pharmaceutical composition is prepared using ingredients known in the art, including, but not limited to, water, glycols, oils, alcohols, flavoring agents, preservatives, and coloring agents. In some embodiments, a pharmaceutical composition of the present invention is a solid (e.g., a powder, tablet, and/or capsule). In some embodiments, a solid pharmaceutical composition including one or more oligonucleotides is prepared using ingredients known in the art, including, but not limited to, starches, sugars, diluents, granulating agents, lubricants, binders, and disintegrating agents. In certain embodiments, a pharmaceutical composition of the present invention is formulated as a depot preparation. In general, depot preparations are typically longer acting than non-depot preparations. In some embodiments, such preparations are administered by implantation (for example subcutaneously or intramuscularly) or by intramuscular injection. In some embodiments, depot preparations are prepared using suitable polymeric or hydrophobic materials (for example an emulsion in an acceptable oil) or ion exchange resins, or as sparingly soluble derivatives, for example, as a sparingly soluble salt.

In some embodiments, a pharmaceutical composition of the present invention includes a delivery system. Examples of delivery systems include, but are not limited to, exosomes, liposomes, and emulsions. In some embodiments, antisense oligonucleotides described herein may be loaded or packaged in exosomes that specifically target a cell type, tissue, or organ to be treated. Exosomes are small membrane-bound vesicles of endocytic origin that are released into the extracellular environment following fusion of mutivesicular bodies with the plasma membrane. Exosome production has been described for many immune cells including B cells, T cells, and dendritic cells, Techniques used to load a therapeutic compound (i.e., an antisense oligonucleotide described herein) into exosomes are known in the art and described in, e.g., U.S. Patent Publication Nos. US 20130053426 and US 20140348904, and International Patent Publication No. WO 2015002956, which are incorporated herein by reference. In some embodiments, therapeutic compounds may be loaded into exosomes by electroporation or the use of a transfection reagent (i.e., cationic liposomes). In some embodiments, an exosome-producing cell can be engineered to produce the exosome and load it with the therapeutic compound (i.e., an antisense oligonucleotide described herein). For example, exosomes may be loaded by transforming or transfecting an exosome-producing host cell with a genetic construct that expresses the therapeutic compound (i.e., an antisense oligonucleotide described herein), such that the therapeutic compound is taken up into the exosomes as the exosomes are produced by the host cell. In some embodiments, an exosome-targeted protein in the exosome-producing cell may bind (i.e., non-covalently) to the therapeutic compound. Various targeting moieties may be introduced into exosomes, so that the exosomes can be targeted to a selected cell type, tissue, or organ. Targeting moieties may bind to cell-surface receptors or other cell-surface proteins or peptides that are specific to the targeted cell type, tissue, or organ. In some embodiments, exosomes have a targeting moiety expressed on their surface. In some embodiments, the targeting moiety expressed on the surface of exosomes is fused to an exosomal transmembrane protein. Techniques of introducing targeting moieties to exosomes are known in the art and described in, e.g., U.S. Patent Publication Nos. US 20130053426 and US 20140348904, and International Patent Publication No. WO 2015002956, which are incorporated herein by reference.

Certain delivery systems are useful for preparing certain pharmaceutical compositions including those including hydrophobic compounds. In some embodiments, certain organic solvents such as dimethylsulfoxide are used. In some embodiments, a pharmaceutical composition of the present invention includes one or more tissue-specific delivery molecules designed to deliver the one or more pharmaceutical agents of the present invention to specific tissues or cell types. For example, in certain embodiments, pharmaceutical compositions include liposomes coated with a tissue-specific antibody. In some embodiments, a pharmaceutical composition of the present invention includes a sustained-release system. A non-limiting example of such a sustained-release system is a semi-permeable matrix of solid hydrophobic polymers. In some embodiments, sustained-release systems may, depending on their chemical nature, release pharmaceutical agents over a period of hours, days, weeks or months.

In some embodiments, a pharmaceutical agent is a sterile lyophilized antisense oligonucleotide that is reconstituted with a suitable diluent, e.g., sterile water for injection. The reconstituted product is administered as a subcutaneous injection or as an intravenous infusion after dilution into saline. In some embodiments, the lyophilized drug product consists of the antisense oligonucleotide which has been prepared in water for injection, adjusted to pH 7.0-9.0 with acid or base during preparation, and then lyophilized. The lyophilized antisense oligonucleotide may be 5-800 mg of the antisense oligonucleotide. It is understood that this encompasses 5, 10, 15, 20, 25, 50, 75, 100, 125, 150, 175, 200, 225, 250, 275, 300, 325, 350, 375, 425, 450, 475, 500, 525, 550, 575, 600, 625, 650, 675, 700, 725, 750, 775, and 800 mg of lyophilized antisense oligonucleotide. The lyophilized drug product may be packaged in a 2 mL Type I, clear glass vial (ammonium sulfate-treated), stoppered with a bromobutyl rubber closure and sealed with an aluminum FLIP-OFF® overseal.

In some embodiments, a pharmaceutical composition is prepared for gene therapy. In some embodiments, the pharmaceutical composition for gene therapy is in an acceptable diluent, or includes a slow release matrix in which the gene delivery vehicle is imbedded. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

In some embodiments, a pharmaceutical composition of the present invention is prepared for oral administration. In some embodiments, a pharmaceutical composition is formulated by combining one or more antisense oligonucleotides with one or more pharmaceutically acceptable carriers and excipients. Certain of such carriers and excipients enable pharmaceutical compositions to be formulated as tablets, pills, dragees, capsules, liquids, gels, syrups, slurries, and suspensions, for oral ingestion by a subject. In some embodiments, pharmaceutical compositions for oral use are obtained by mixing oligonucleotide and one or more solid excipients. Suitable carriers and excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In some embodiments, such a mixture is optionally ground and auxiliaries are optionally added. In some embodiments, pharmaceutical compositions are formed to obtain tablets or dragee cores. In some embodiments, disintegrating agents (e.g., cross-linked polyvinyl pyrrolidone, agar, or alginic acid or a salt thereof, such as sodium alginate) are added.

In some embodiments, a pharmaceutical composition is prepared for administration by injection (e.g., intravenous, subcutaneous, intramuscular, etc.). In some embodiments, a pharmaceutical composition includes a carrier and is formulated in aqueous solution, such as water or physiologically compatible buffers such as PBS, Hank's solution, Ringer's solution, or physiological saline buffer. Examples of solvents suitable for use in pharmaceutical compositions for injection include, but are not limited to, lipophilic solvents and fatty oils, such as sesame oil, and synthetic fatty acid esters, such as ethyl oleate or triglycerides. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In some embodiments, a pharmaceutical composition is prepared for topical administration. Certain of such pharmaceutical compositions include bland moisturizing bases, such as ointments or creams. Exemplary suitable ointment bases include, but are not limited to, petrolatum, petrolatum plus volatile silicones, lanolin, and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP), and Lubriderm™, available from Pfizer (Morris Plains, N.J.).

### VII. Routes, Dosage, and Administration

In some embodiments, a pharmaceutical composition including an antisense oligonucleotide described herein is used for the preparation of a medicament for inhibiting the expression of a mutant ACVR1 gene in a subject. In some embodiments, a pharmaceutical composition including an antisense oligonucleotide described herein is used for the preparation of a medicament for treating a subject having a disease or condition (e.g., FOP or DIPG) associated with the expression of a mutant ACVR1 gene. Pharmaceutical compositions including an antisense oligonucleotide described herein may be formulated for parenteral administration, e.g., intravenous administration, subcutaneous administration, intramuscular administration, intra-arterial administration, intrathecal administration, or intraperitoneal administration. Other administration routes include, but are not limited to, oral, rectal, transmucosal, intestinal, enteral, topical, suppository, through inhalation, intranasal, and intraocular administration. The pharmaceutical composition may also be formulated for, or administered via, e.g., oral, nasal, spray, aerosol, rectal, or vaginal administration. In some embodiments, pharmaceutical intrathecals are administered to achieve local rather than systemic exposures. For example, pharmaceutical compositions may be injected directly in the area of desired effect. For injectable formulations, various effective pharmaceutical carriers are known in the art, see, e.g., ASHP Handbook on Injectable Drugs, Trissel, 18th ed. (2014).

In some embodiments, administration of an antisense oligonucleotide described herein targeted to a mutant ACVR1 gene is parenteral administration. Parenteral administration may be intravenous or subcutaneous administration. In some embodiments, administration of an antisense oligonucleotide described herein targeted to a mutant ACVR1 gene is intravenous or subcutaneous administration. Administration may include a single dose or multiple doses of an antisense oligonucleotide targeted to a mutant ACVR1 gene. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, PBS, and cell culture media (e.g., Dulbecco's Modified Eagle Medium (DMEM), α-Modified Eagles Medium (α-MEM), F-12 medium). Formulation methods are known in the art, see e.g., Therapeutic Peptides and Proteins: Formulation, Processing and Delivery Systems, Banga, 3rd ed. (2015). In some embodiments, pharmaceutical compositions for injection are presented in unit dosage form, e.g., in ampoules or in multi-dose containers.

In some embodiments, one or more pharmaceutical compositions described herein are co-administered with one or more other pharmaceutical agents. In some embodiments, such one or more other pharmaceutical agents are designed to treat the same disease or condition as the one or more pharmaceutical compositions of the invention. In some embodiments, such one or more other pharmaceutical agents are designed to treat a different disease or condition as the one or more pharmaceutical compositions of the invention. In some embodiments, such one or more other pharmaceutical agents are designed to treat an undesired effect of one or more pharmaceutical compositions of the invention. In some embodiments, one or more pharmaceutical compositions of the invention and one or more other pharmaceutical agents are administered at the same time. In some embodiments, one or more pharmaceutical compositions of the invention and one or more other pharmaceutical agents are administered at different times. In some embodiments, one or more pharmaceutical compositions of the invention and one or more other pharmaceutical agents are prepared together in a single formulation. In some embodiments, one or more pharmaceutical compositions of the invention and one or more other pharmaceutical agents are prepared separately.

In some embodiments, a pharmaceutical composition described herein is administered in the form of a dosage unit (e.g., tablet, capsule, bolus, etc.). In some embodiments, a pharmaceutical compositions includes an antisense oligonucleotide in a dose selected from 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In some embodiments, a pharmaceutical composition described herein includes a dose of an antisense oligonucleotide selected from 25 mg, 50 mg, 75 mg, 100 mg, 150 mg, 200 mg, 250 mg, 300 mg, 350 mg, 400 mg, 500 mg, 600 mg, 700 mg, and 800 mg. In some embodiments, a pharmaceutical composition includes a dose of oligonucleotide selected from 50 mg, 100 mg, 150 mg, 200 mg, 250 mg, 300 mg, and 400 mg. In some embodiments, a pharmaceutical composition includes an antisense oligonucleotide in a dose ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 50 mg/kg and, in a more specific embodiment, about 1 to about 5 mg/kg.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). In some embodiments, the dose is administered at intervals ranging from more than once per day, once per day, once per week, twice per week, three times per week, four times per week, five times per week, six times per week, once per month to once per three months, for as long as needed to sustain the desired effect. The timing between administrations may decrease as the medical condition improves or increase as the health of the patient declines. The dosage may be adapted by the physician in accordance with conventional factors such as the extent of the disease and different parameters of the subject.

### VIII. Indications and Methods of Treatment

Fibrodysplasia ossificans progressiva (FOP) is a disorder in which muscle tissue and connective tissue, such as tendons and ligaments, are gradually replaced by ossified bone, forming bone outside the skeleton (extra-skeletal or heterotopic bone) that constrains movement. Extra-skeletal bone formation causes progressive loss of mobility as the joints become affected. Any trauma to the muscles of an individual with FOP, such as a fall, muscle-related diseases, or invasive medical procedures, may trigger episodes of muscle swelling and inflammation (myositis) followed by more rapid ossification in the injured area.

The causative genetic mutation in FOP is a guanine to adenosine substitution (c.617G>A) in the activin A receptor type-1 (ACVR1) gene, which leads to an arginine to histidine substitution at amino acid position 206 in the ACVR1 protein. The sequence of a human, wild-type ACVR1 gene is shown in NCBI Gene ID NO: 90. The ACVR1 protein is found in many tissues of the body including skeletal muscle and cartilage and helps to control the growth and development of the bones and muscles, including the gradual replacement of cartilage by bone (ossification) that occurs in normal skeletal maturation. The ACVR1 protein transduces signals through bone morphogenetic proteins (BMPs). A mutation in the ACVR1 gene may change the structure of the receptor under certain conditions and disrupt mechanisms that control the receptor's activity. As a result, the receptor may be constitutive activated, which causes overgrowth of bone and cartilage and fusion of joints, resulting in the signs and symptoms of FOP.

Diffuse Intrinsic Pontine Glioma (DIPG) is a fatal brain cancer that arises in the brainstem of children with no effective treatment and near 100% fatality. Approximately 20% of DIPG have ACVR1 genetic mutations. The guanine to adenosine substitution (c.617G>A) in the ACVR1 gene is one of several causative mutations found in DIPG. The mutation in the mutant ACVR1 gene is constitutively activating, leading to downstream protein phosphorylation and increased expression of downstream BMP signaling proteins.

The antisense oligonucleotides of the invention may be of therapeutic benefit in diseases or disorders associated with the expression of a mutant ACVR1 gene, in which the mutant ACVR1 gene has the mutation c.617G>A, which causes an arginine to histidine substitution at amino acid position 206 in the mutant ACVR1 protein. The antisense oligonucleotide of the invention hybridizes to the region of the mutant ACVR1 gene containing the mutant adenine base and activates RNaseH cleavage of the mutant ACVR1 gene. The ability of the antisense oligonucleotides to hybridize to the mutant ACVR1 gene and activate RNaseH cleavage of the mutant gene prevents the expression of the mutant ACVR1 protein and offers methods of treating diseases and disorders (e.g., FOP and DIPG) that are associated with or caused by the expression of the mutant ACVR1 gene (i.e., the mutant ACVR1 gene having the mutation c.617G>A).

The invention features a method of inhibiting the expression of a mutant ACVR1 gene in a subject. The method includes administering to the subject a therapeutically effective amount of an antisense oligonucleotide described herein or a pharmaceutical composition containing an antisense oligonucleotide described herein, wherein the mutant ACVR1 gene has the mutation c.617G>A.

The invention also features a method of treating a subject having a disease or condition associated with the expression of a mutant ACVR1 gene. The method includes administering to the subject a therapeutically effective amount of an antisense oligonucleotide described herein or a pharmaceutical composition containing an antisense oligonucleotide described herein, wherein the mutant ACVR1 gene has the mutation c.617G>A and wherein the antisense oligonucleotide inhibits the expression of the mutant ACVR1 gene.

In some embodiments of the methods of the invention, the disease associated with the expression of a mutant ACVR1 protein is FOP. In some embodiments of the methods of the invention, the disease associated with the expression of a mutant ACVR1 protein is DIPG. In some embodiments of the methods of the invention, the antisense oligonucleotide preferentially hybridizes to the mutant ACVR1 gene having the mutation c.617G>A over a wild-type ACVR1 gene.

### EXAMPLES

### Example 1—In Vitro Screening

The antisense oligonucleotides were screened in vitro to determine their ability to inhibit the expression of the mutant ACVR1 gene and their potency against mutant and wild-type forms of the ACVR1 gene. Fibroblasts from FOP patients (Coriell cell line GM00783) were used in the in vitro experiments. These cells are expressing both the wild-type and mutant forms of the ACVR1 gene. The cells were seeded at 10,000 cells per well in a single 96 well culture plate using 150 μL growth medium and placed into the incubator overnight. The cells were transfected with different concentrations (concentrations between 25 nM and 200 nM) of an antisense oligonucleotide having a sequence of any one of SEQ ID NOs: 13-20. Sequences of SEQ ID NOs: 13-20 are as follows:

SEQ ID NO: 13:
CTGGTGAGCCACTGTT;
SEQ ID NO: 14:
TCTGGTGAGCCACTGT;
SEQ ID NO: 15:
ATCTGGTGAGCCACTG;
SEQ ID NO: 16:
AATCTGGTGAGCCACT;
SEQ ID NO: 17:
TAATCTGGTGAGCCAC;
SEQ ID NO: 18:
GTAATCTGGTGAGCCA;
SEQ ID NO: 19:
TGTAATCTGGTGAGCC;
and
SEQ ID NO: 20:
GTGTAATCTGGTGAGC.

In a first set of experiments, the antisense oligonucleotides were solubilized in 100 μL nuclease free water as per LNA longRNA GapmeR instruction manual (v2.0) to generate 50 μM stock solutions. Two dilutions of antisense oligonucleotides in serum free medium were prepared (500 nM and 4 μM) to generate a final cell treatment concentration of 25 and 200 nM. A 1:1 mix of diluted Lipofectamine 2000 in serum free medium and antisense oligonucleotides dilutions were generated for complex formation. The antisense oligonucleotide and Lipofectamine mixtures were incubated at room temperature for 5 minutes. Plating medium was removed from the plates of FOP cells and replaced with 90 μL growth medium per well. Ten (10) μL of each antisense nucleotides/Lipofectamine mixture was added to the appropriate wells and the cells placed into the incubator overnight. Twenty-four hours post-transfection, the cells were washed with 50 μL ice-cold PBS and lysed in 40 μL of lysis buffer containing 1:100 dilution of DNase I. Lysis reaction was incubated at room temperature for 5 minutes with shaking. Four (4) μL of Stop reagent was added per well and mixed on plate shaker for 2 minutes at room temperature. Plates containing cell lysates were frozen at −20° C. until RT-PCR analysis.

The relative levels of mutant and wild-type ACVR1 gene and of a housekeeping gene (GAPDH) were determined by allele-specific RT-PCR and were amplified in separate wells. PCR primers and one-step RT-PCR reaction conditions were as follows: forward and reverse primers (for wild-type ACVR1 gene detection: forward primer: 5′-TGGTACAAAGAACAGTGGCTAG-3′ and reverse primer: 5′-CCATACCTGCCTTTCCCGA-3′; for mutant ACVR1 gene detection: forward primer: 5′-TGGTACAAAGAACAGTGGCTTA-3′ and reverse primer: 5′-CCATACCTGCCTTTCCCGA-3′; for GAPDH gene detection: forward primer: 5′-AGATCATCAGCAATGCCTCCTG-3′ and reverse primer: 5′-ATGGCATGGACTGTGGTCATG-3′), cell lysates (1:20 dilution), and Cells-to-CT™ 1-Step Power SYBR® Green Kit (Life Technologies, #A25600). PCR reactions for allele-specific detection were performed at extension temperature of 63° C.

The RT-PCR data were analyzed using the 2^{ΔΔCT} method and were presented as the fold change in wild-type and mutant ACVR1 gene expression normalized to the housekeeping gene GAPDH and relative to the untreated. Tabulated results are presented in Tables 1 to 4 (ASO, antisense oligonucleotide; UNT, untreated; * value identified as outlier and excluded from the 2^{-ddCT} method analysis; Und, value undetermined).

TABLE 1
Antisense oligonucleotides at 25 nM (raw data)

| | Mutant ACVR1 | | | Wild Type ACVR1 | | | GAPDH | | |
|---|---|---|---|---|---|---|---|---|---|
| ASO | CT | | | CT | | | CT | | |
| SEQ ID NO: 13 | 34.183 | 33.636 | 35.408 | 34.211 | 32.465 | 32.454 | 23.990 | 24.658 | 25.066 |
| SEQ ID NO: 14 | 35.114 | 33.589 | 35.463 | 34.698 | 32.949 | 33.521 | 25.593 | 24.583 | 24.388 |
| SEQ ID NO: 15 | 33.864 | 32.770 | 34.756 | 32.574 | 32.116 | 34.135 | 24.014 | 23.644 | 23.968 |
| SEQ ID NO: 16 | 35.153 | 33.747 | 33.449 | 34.417 | 32.835 | 32.393 | 25.862 | 23.726 | 23.572 |
| SEQ ID NO: 17 | 34.618 | 32.955 | 35.601 | 33.219 | 33.222 | 33.205 | 25.597 | 23.796 | 23.802 |
| SEQ ID NO: 18 | 35.610 | 34.669 | 32.990 | 33.211 | 34.415 | 32.875 | 26.510 | 24.746 | 23.597 |
| SEQ ID NO: 19 | 36.789 | 33.737 | 33.816 | *37.623 | 32.668 | 32.426 | 25.595 | 23.506 | 23.197 |
| SEQ ID NO: 20 | 34.846 | 33.594 | 34.007 | 33.351 | 33.851 | 33.003 | 25.706 | 23.541 | 23.031 |
| UNT | *32.333 | 34.761 | 33.025 | 31.754 | 32.823 | 32.855 | 23.255 | 23.553 | 23.984 |
| | *37.021 | 34.144 | 32.835 | *33.438 | *34.163 | 32.936 | 25.765 | 25.350 | 23.399 |

TABLE 2
Antisense oligonucleotides at 25 nM (percentage
change in wild-type and mutant ACVR1 gene normalized
expression relative to the untreated)

| | Mutant ACVR1 | Wild Type ACVR1 |
|---|---|---|
| ASO | % UNT | % UNT |
| SEQ ID NO: 13 | 78 | 94 |
| SEQ ID NO: 14 | 76 | 71 |
| SEQ ID NO: 15 | 73 | 62 |
| SEQ ID NO: 16 | 84 | 73 |
| SEQ ID NO: 17 | 70 | 74 |
| SEQ ID NO: 18 | 100 | 89 |
| SEQ ID NO: 19 | 43 | 95 |
| SEQ ID NO: 20 | 67 | 52 |
| UNT | 100 | 100 |

TABLE 3
Antisense oligonucleotides at 200 nM (raw data)

| | Mutant ACVR1 | | | Wild Type ACVR1 | | | GAPDH | | |
|---|---|---|---|---|---|---|---|---|---|
| ASO | CT | | | CT | | | CT | | |
| SEQ ID NO: 13 | 34.658 | 35.294 | 35.769 | 32.846 | 33.904 | 32.447 | 24.214 | 25.007 | 25.044 |
| SEQ ID NO: 14 | 33.601 | 34.700 | 34.619 | 33.609 | 33.955 | 33.023 | 24.087 | 24.973 | 24.286 |
| SEQ ID NO: 15 | 34.478 | 35.320 | 35.824 | 33.624 | 34.725 | 34.749 | 23.992 | 24.578 | 24.256 |
| SEQ ID NO: 16 | 35.144 | 34.374 | 34.586 | 33.566 | 34.675 | Und | 23.947 | 24.107 | 24.169 |
| SEQ ID NO: 17 | 35.653 | 36.526 | Und | 33.856 | 33.965 | 31.563 | 24.249 | 24.364 | 24.223 |
| SEQ ID NO: 18 | 35.333 | 35.561 | 34.734 | 33.273 | 33.981 | 34.427 | 23.910 | 23.828 | Und |
| SEQ ID NO: 19 | 34.323 | 36.512 | 36.242 | 34.534 | 33.962 | 33.930 | 23.359 | 23.594 | 23.237 |
| SEQ ID NO: 20 | 35.442 | 34.765 | *37.443 | 33.529 | 33.913 | *35.566 | 23.111 | 23.127 | 22.986 |
| UNT | *32.333 | 34.761 | 33.025 | 31.754 | 32.823 | 32.855 | 23.255 | 23.553 | 23.984 |
| | *37.021 | 34.144 | 32.835 | *33.438 | *34.163 | 32.936 | 25.765 | 25.350 | 23.399 |

TABLE 4
Antisense oligonucleotides at 200 nM (percentage
change in wild-type and mutant ACVR1 gene normalized
expression relative to the untreated)

| | Mutant ACVR1 | Wild Type ACVR1 |
|---|---|---|
| ASO | % UNT | % UNT |
| SEQ ID NO: 13 | 50 | 104 |
| SEQ ID NO: 14 | 77 | 61 |
| SEQ ID NO: 15 | 36 | 30 |
| SEQ ID NO: 16 | 45 | 31 |
| SEQ ID NO: 17 | 20 | 72 |
| SEQ ID NO: 18 | 27 | 32 |
| SEQ ID NO: 19 | 14 | 19 |
| SEQ ID NO: 20 | 17 | 21 |
| UNT | 100 | 100 |

In a subsequent experiment, optimized transfection conditions and two housekeeping genes were used for normalization. The antisense oligonucleotides were solubilized in 100 μL nuclease free water as per LNA long RNA GapmeR instruction manual (v2.0) to generate 50 μM stock solutions. One dilution of antisense oligonucleotides in serum free medium was prepared (1.5 μM) to generate a final cell treatment concentration of 75 nM. A 1:1 mix of diluted Lipofectamine 2000 in serum free medium and antisense oligonucleotides dilutions were generated for complex formation. The antisense oligonucleotide and Lipofectamine mixtures were incubated at room temperature for 5 minutes. Plating medium was removed from the plates of FOP cells and replaced with 90 μL growth medium per well. Ten (10) μL of each antisense nucleotides/Lipofectamine mixture was added to the appropriate wells and the cells placed into the incubator for 5 hours. After the 5 hour transfection time, the transfection medium was removed and fresh medium was applied to each well. The cells were returned to the incubator for 48 hours. Forty-height (48) hours post-transfection, the cells were washed with 50 μL ice-cold PBS and lysed in 40 μL of lysis buffer containing 1:100 dilution of DNase I. Lysis reaction was incubated at room temperature for 5 minutes with shaking. Four (4) μL of Stop reagent was added per well and mixed on plate shaker for 2 minutes at room temperature. Plates containing cell lysates were frozen at −20° C. until RT-PCR analysis.

The relative levels of mutant and wild-type ACVR1 gene and of two housekeeping genes

(GAPDH and 18s) were determined by allele-specific RT-PCR and were amplified in separate wells. PCR primers and one-step RT-PCR reaction conditions were as follows: forward and reverse primers (for wild-type ACVR1 gene detection: forward primer: 5′-TGGTACAAAGAACAGTGGCTAG-3′ and reverse primer: 5′-CCATACCTGCCTTTCCCGA-3′; for mutant ACVR1 gene detection: forward primer: 5′-TGGTACAAAGAACAGTGGCTTA-3′ and reverse primer: 5′-CCATACCTGCCTTTCCCGA-3′; for GAPDH gene detection: forward primer: 5′-AGATCATCAGCAATGCCTCCTG-3′ and reverse primer: 5′-ATGGCATGGACTGTGGTCATG-3′; for 18s ribosomal RNA detection: QuantiTect Primer Assay, Qiagen, Hs_RRN18S_1_SG, cat #QT00199367), cell lysates (1:20 dilution), and Cells-to-CT™ 1-Step Power SYBR® Green Kit (Life Technologies, #A25600). PCR reactions for allele-specific detection were performed at extension temperature of 63° C.

The RT-PCR data were analyzed using the 2^{ΔΔCT} method and were presented as the fold change in wild-type and mutant ACVR1 gene expression normalized to the housekeeping genes GAPDH and 18s ribosomal RNA, and relative to the untreated. Tabulated results are presented in Tables 5 and 6 (ASO, antisense oligonucleotide; UNT, untreated; * value identified as outlier and excluded from the 2^{-ddCT} method analysis).

TABLE 5
Antisense oligonucleotides at 75 nM (raw data)

| | Mutant ACVR1 | | | Wild Type ACVR1 | | | GAPDH | | | 18S | | |
|---|---|---|---|---|---|---|---|---|---|---|---|---|
| ASO | CT | | | CT | | | CT | | | CT | | |
| SEQ ID NO: 13 | 34.987 | 34.505 | 34.628 | 34.259 | 33.287 | 33.931 | 24.297 | 23.889 | 23.913 | 8.489 | 8.400 | 8.065 |
| SEQ ID NO: 14 | 34.227 | 35.195 | 33.613 | 32.843 | 33.570 | 33.412 | 23.408 | 24.344 | 23.671 | 7.914 | *16.236 | 7.972 |
| SEQ ID NO: 15 | 33.551 | 33.719 | 34.539 | 32.799 | 32.647 | 34.066 | 23.262 | 24.301 | 23.811 | 8.008 | 8.479 | 7.963 |
| SEQ ID NO: 16 | 35.417 | 34.294 | 33.143 | 33.407 | 32.580 | 33.662 | 23.319 | 23.340 | 23.747 | 7.799 | 8.162 | 7.763 |
| SEQ ID NO: 17 | 33.952 | 33.806 | 33.251 | 32.262 | 32.115 | 32.653 | 23.162 | 23.483 | 23.594 | 7.711 | 8.099 | 7.876 |
| SEQ ID NO: 18 | 34.006 | 33.269 | 33.934 | 32.882 | 33.213 | 32.909 | 23.924 | 24.242 | 24.626 | 8.291 | 8.455 | 8.561 |
| SEQ ID NO: 19 | 34.009 | 35.506 | 34.969 | 33.778 | 32.986 | 32.468 | 23.047 | 23.783 | 23.987 | 8.269 | 8.543 | 8.367 |
| SEQ ID NO: 20 | 33.470 | 33.658 | *30.826 | 32.526 | 33.172 | *29.728 | 22.922 | 22.862 | 24.538 | 7.753 | 8.364 | 9.501 |
| UNT | 33.130 | 33.520 | 34.799 | 32.335 | 32.340 | 31.940 | 23.137 | 23.760 | 25.541 | 8.732 | 9.022 | 9.605 |
| | 33.578 | 33.154 | 32.714 | 32.420 | 32.201 | 33.689 | 23.135 | 23.595 | 23.760 | 8.272 | 8.417 | 8.288 |

TABLE 6
Antisense oligonucleotides at 75 nM (percentage
change in wild-type and mutant ACVR1 gene normalized
expression relative to the untreated)

| | Mutant ACVR1 | Wild Type ACVR1 |
|---|---|---|
| ASO | % UNT | % UNT |
| SEQ ID NO: 13 | 40 | 37 |
| SEQ ID NO: 14 | 42 | 44 |
| SEQ ID NO: 15 | 59 | 50 |
| SEQ ID NO: 16 | 38 | 40 |
| SEQ ID NO: 17 | 57 | 72 |
| SEQ ID NO: 18 | 88 | 74 |
| SEQ ID NO: 19 | 33 | 55 |
| SEQ ID NO: 20 | 78 | 64 |
| UNT | 100 | 100 |

### Example 2—Evaluation of Antisense Oligonucleotides in Transgenic Mouse Model of FOP

To evaluate the ability of the antisense oligonucleotides to inhibit the expression of the mutant ACVR1 gene in vivo, transgenic mice that express the mutant, human ACVR1 gene may be used. The mice may be divided into four groups. The groups are injected (i.e., subcutaneously) with PBS, negative control (or left untreated), the best antisense oligonucleotide from the in vitro screen, and the back-up antisense oligonucleotide. The effect of these treatments on FOP endpoints may be determined. FOP endpoints may include prevention or reduction of signs or symptoms of FOP, including soft tissue swelling, pain, stiffness, decrease range of motion, redness, and warmth, prevention or reduction of heterotopic ossification, maintenance of range of motion and functional ability, preservation of quality of life, and improved survival. To better understand the results, mutant ACVR1 mRNA levels in tissues may be determined. For example, the tissue with the highest active antisense oligonucleotide distribution may be the liver. If the gene is expressed in the liver, the liver would be an appropriate tissue to use to get read-outs of mutant ACVR1 mRNA levels. It may also be helpful to determine gene expression levels in the bone marrow or any other organ/tissue (i.e., muscle) that is likely to play an important role in the pathogenesis of the disease.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

Other embodiments are within the following claims.

What is claimed is:

1. A single-stranded antisense oligonucleotide that is 12 to 30 nucleosides in length, wherein the antisense oligonucleotide is complementary to an equal length portion of the sequence of TTTCTGGTACAAAGAACAGTGGCTCACCAGATTACACTGTTGGAGTGTGTC (SEQ ID NO: 1) and comprises a contiguous portion that is complementary to the sequence of GCTCACCAG (SEQ ID NO: 2).

2. The antisense oligonucleotide of claim 1, wherein the antisense oligonucleotide comprises at least one modified sugar.

3. The antisense oligonucleotide of claim 2, wherein the modified sugar is selected from the group consisting of a bicyclic sugar, a 2′-O-methoxyethyl modified sugar, a 2′-methoxy modified sugar, a 2′-O-alkyl modified sugar, and an unlocked sugar.

4. The antisense oligonucleotide of claim 3, wherein the bicyclic sugar is a locked sugar.

5. The antisense oligonucleotide of any one of claims 1-4, wherein the antisense oligonucleotide comprises at least one modified internucleoside linkage.

6. The antisense oligonucleotide of claim 5, wherein the modified internucleoside linkage is a phosphorothioate internucleoside linkage.

7. The antisense oligonucleotide of claim 5 or 6, wherein the antisense oligonucleotide has phosphorothioate internucleoside linkages between neighboring nucleosides throughout the length of the antisense oligonucleotide.

8. The antisense oligonucleotide of any one of claims 1-7, wherein the antisense oligonucleotide comprises at least one modified nucleobase.

9. The antisense oligonucleotide of claim 8, wherein the modified nucleobase is selected from the group consisting of 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halouracil, 5-bromouracil, 5-trifluoromethyluracil, 5-halocytosine, 5-bromocytosine, 5-trifluoromethylcytosine, 7-methylguanine, 7-methyladenine, 2-fluoroadenine, 2-aminoadenine, 8-azaguanine, 8-azaadenine, 7-deazaguanine, 7-deazaadenine, 3-deazaguanine, and 3-deazaadenine.

10. The antisense oligonucleotide of claim 9, wherein the modified nucleobase is 5-methylcytosine.

11. The antisense oligonucleotide of any one of claims 1-10, wherein the antisense oligonucleotide is a gapmer comprising a gap segment flanked by a 5′ wing segment and a 3′ wing segment.

12. The antisense oligonucleotide of claim 11, wherein the gap segment comprises 6 to 10 2′-deoxyribonucleosides.

13. The antisense oligonucleotide of claim 11 or 12, wherein each of the 5′ and 3′ wing segments comprises 2 to 6 nucleosides and at least one modified sugar.

14. The antisense oligonucleotide of claim 13, wherein the modified sugar is selected from the group consisting of a bicyclic sugar, a 2′-O-methoxyethyl modified sugar, a 2′-methoxy modified sugar, a 2′-O-alkyl modified sugar, and an unlocked sugar.

15. The antisense oligonucleotide of claim 14, wherein the modified sugar is a locked sugar.

16. The antisense oligonucleotide of any one of claims 13-15, wherein each of the 5′ and 3′ wing segments comprises 2 to 6 nucleosides each having a locked sugar.

17. The antisense oligonucleotide of claim 15 or 16, wherein the locked sugar has the 2′-oxygen linked to the 4′ ring carbon by way of a methylene.

18. The antisense oligonucleotide of any one of claims 11-17, wherein the 5′ wing segment comprises at least one modified nucleobase.

19. The antisense oligonucleotide of any one of claims 11-18, wherein the 3′ wing segment comprises at least one modified nucleobase.

20. The antisense oligonucleotide of claim 18 or 19, wherein the modified nucleobase is 5-methylcytosine.

21. The antisense oligonucleotide of any one of claims 11-20, wherein the gapmer has phosphorothioate internucleoside linkages between neighboring nucleosides throughout the length of the gapmer.

22. The antisense oligonucleotide of any one of claims 11-21, wherein the gap segment comprises 8 2′-deoxyribonucleosides, each of the 5′ and 3′ wing segments comprises 4 nucleosides each having a locked sugar, and the gapmer has phosphorothioate internucleoside linkages between neighboring nucleosides throughout the length of the gapmer.

23. The antisense oligonucleotide of claim 22, wherein the locked sugar has the 2′-oxygen linked to the 4′ ring carbon by way of a methylene.

24. The antisense oligonucleotide of any one of claims 1-23, wherein the antisense oligonucleotide is 12 to 20 nucleosides in length.

25. The antisense oligonucleotide of claim 24, wherein the antisense oligonucleotide is 16 nucleosides in length.

26. The antisense oligonucleotide of any one of claims 1-25, wherein the antisense oligonucleotide comprises the sequence of CTGGTGAGC (SEQ ID NO: 3).

27. An antisense oligonucleotide that is 16 nucleosides in length, wherein each of nucleosides 1-4 and 13-16 has a locked sugar, each of nucleosides 5-12 is a 2′-deoxyribonucleoside, the antisense oligonucleotide has phosphorothioate internucleoside linkages between neighboring nucleosides throughout the length of the antisense oligonucleotide, and the antisense oligonucleotide is complementary to an equal length portion of the sequence of AACAGTGGCTCACCAGATTACAC (SEQ ID NO: 4) and comprises a contiguous portion that is complementary to the sequence of GCTCACCAG (SEQ ID NO: 2).

28. The antisense oligonucleotide of claim 27, wherein the locked sugar has the 2′-oxygen linked to the 4′ ring carbon by way of a methylene.

29. The antisense oligonucleotide of claim 27 or 28, wherein the antisense oligonucleotide is 16 nucleosides in length and is complementary to a sequence of any one of AACAGTGGCTCACCAG (SEQ ID NO: 5), ACAGTGGCTCACCAGA (SEQ ID NO: 6), CAGTGGCTCACCAGAT (SEQ ID NO: 7), AGTGGCTCACCAGATT (SEQ ID NO: 8), GTGGCTCACCAGATTA (SEQ ID NO: 9), TGGCTCACCAGATTAC (SEQ ID NO: 10), GGCTCACCAGATTACA (SEQ ID NO: 11), and GCTCACCAGATTACAC (SEQ ID NO: 12).

30. The antisense oligonucleotide of any one of claims 27-29, wherein the antisense oligonucleotide has a sequence of any one of CTGGTGAGCCACTGTT (SEQ ID NO: 13), TCTGGTGAGCCACTGT (SEQ ID NO: 14), ATCTGGTGAGCCACTG (SEQ ID NO: 15), AATCTGGTGAGCCACT (SEQ ID NO: 16), TAATCTGGTGAGCCAC (SEQ ID NO: 17), GTAATCTGGTGAGCCA (SEQ ID NO: 18), TGTAATCTGGTGAGCC (SEQ ID NO: 19), and GTGTAATCTGGTGAGC (SEQ ID NO: 20).

31. The antisense oligonucleotide of claim 30, wherein the nucleobase at position 1 of the sequence of CTGGTGAGCCACTGTT (SEQ ID NO: 13) is 5-methylcytosine.

32. The antisense oligonucleotide of claim 30, wherein the nucleobases at positions 2 and 13 of the sequence of TCTGGTGAGCCACTGT (SEQ ID NO: 14) are 5-methylcytosines.

33. The antisense oligonucleotide of claim 30, wherein the nucleobases at positions 3 and 14 of the sequence of ATCTGGTGAGCCACTG (SEQ ID NO: 15) are 5-methylcytosines.

34. The antisense oligonucleotide of claim 30, wherein the nucleobases at positions 4, 13, and 15 of the sequence of AATCTGGTGAGCCACT (SEQ ID NO: 16), are 5-methylcytosines.

35. The antisense oligonucleotide of claim 30, wherein the nucleobases at positions 13, 14, and 16 of the sequence of TAATCTGGTGAGCCAC (SEQ ID NO: 17) are 5-methylcytosines.

36. The antisense oligonucleotide of claim 30, wherein the nucleobases at positions 14 and 15 of the sequence of GTAATCTGGTGAGCCA (SEQ ID NO: 18) are 5-methylcytosines.

37. The antisense oligonucleotide of claim 30, wherein the nucleobases at positions 15 and 16 of the sequence of TGTAATCTGGTGAGCC (SEQ ID NO: 19) are 5-methylcytosines.

38. The antisense oligonucleotide of claim 30, wherein the nucleobase at position 16 of the sequence of GTGTAATCTGGTGAGC (SEQ ID NO: 20) is 5-methylcytosine.

39. The antisense oligonucleotide of any one of claims 1-38, wherein the antisense oligonucleotide preferentially hybridizes to a mutant activin A receptor type-1 (ACVR1) gene over a wild-type ACVR1 gene, wherein the mutant ACVR1 gene has the mutation c.617G>A.

40. A pharmaceutical composition comprising an antisense oligonucleotide of any one of claims 1-39 and one or more pharmaceutically acceptable carriers or excipients.

41. A method of inhibiting the expression of a mutant ACVR1 gene in a subject, comprising administering to the subject a therapeutically effective amount of an antisense oligonucleotide of any one of claims 1-39 or a pharmaceutical composition of claim 40, wherein the mutant ACVR1 gene has the mutation c.617G>A.

42. A method of treating a subject having a disease or condition associated with the expression of a mutant ACVR1 gene, comprising administering to the subject a therapeutically effective amount of an antisense oligonucleotide of any one of claims 1-39 or a pharmaceutical composition of claim 40, wherein the mutant ACVR1 gene has the mutation c.617G>A and wherein the antisense oligonucleotide inhibits the expression of the mutant ACVR1 gene.

43. The method of claim 42, wherein the disease is fibrodysplasia ossificans progressiva (FOP).

44. The method of claim 42, wherein the disease is diffuse intrinsic pontine glioma (DIPG).

45. A method of preventing or reducing heterotopic ossification in a subject having FOP, comprising administering to the subject a therapeutically effective amount of an antisense oligonucleotide of any one of claims 1-39 or a pharmaceutical composition of claim 40, wherein the subject has a mutant ACVR1 gene, wherein the mutant ACVR1 gene has the mutation c.617G>A, and wherein the antisense oligonucleotide inhibits the expression of the mutant ACVR1 gene.

46. The method of any one of claims 41-45, wherein the antisense oligonucleotide preferentially hybridizes to the mutant ACVR1 gene over a wild-type ACVR1 gene.


SEQUENCE LISTING (20 sequences)

SEQ ID NO: 1 - DNA, 51 bp, Artificial Sequence
  Synthetic Construct
tttctggtacaaagaacagtggctcaccagattacactgttggagtgtgtc

SEQ ID NO: 2 - DNA, 9 bp, Artificial Sequence
  Synthetic Construct
gctcaccag

SEQ ID NO: 3 - DNA, 9 bp, Artificial Sequence
  Synthetic Construct
ctggtgagc

SEQ ID NO: 4 - DNA, 23 bp, Artificial Sequence
  Synthetic Construct
aacagtggctcaccagattacac

SEQ ID NO: 5 - DNA, 16 bp, Artificial Sequence
  Synthetic Construct
aacagtggctcaccag

SEQ ID NO: 6 - DNA, 16 bp, Artificial Sequence
  Synthetic Construct
acagtggctcaccaga

SEQ ID NO: 7 - DNA, 16 bp, Artificial Sequence
  Synthetic Construct
cagtggctcaccagat

SEQ ID NO: 8 - DNA, 16 bp, Artificial Sequence
  Synthetic Construct
agtggctcaccagatt

SEQ ID NO: 9 - DNA, 16 bp, Artificial Sequence
  Synthetic Construct
gtggctcaccagatta

SEQ ID NO: 10 - DNA, 16 bp, Artificial Sequence
  Synthetic Construct
tggctcaccagattac

SEQ ID NO: 11 - DNA, 16 bp, Artificial Sequence
  Synthetic Construct
ggctcaccagattaca

SEQ ID NO: 12 - DNA, 16 bp, Artificial Sequence
  Synthetic Construct
gctcaccagattacac

SEQ ID NO: 13 - DNA, 16 bp, Artificial Sequence
  Synthetic Construct
ctggtgagccactgtt

SEQ ID NO: 14 - DNA, 16 bp, Artificial Sequence
  Synthetic Construct
tctggtgagccactgt

SEQ ID NO: 15 - DNA, 16 bp, Artificial Sequence
  Synthetic Construct
atctggtgagccactg

SEQ ID NO: 16 - DNA, 16 bp, Artificial Sequence
  Synthetic Construct
aatctggtgagccact

SEQ ID NO: 17 - DNA, 16 bp, Artificial Sequence
  Synthetic Construct
taatctggtgagccac

SEQ ID NO: 18 - DNA, 16 bp, Artificial Sequence
  Synthetic Construct
gtaatctggtgagcca

SEQ ID NO: 19 - DNA, 16 bp, Artificial Sequence
  Synthetic Construct
tgtaatctggtgagcc

SEQ ID NO: 20 - DNA, 16 bp, Artificial Sequence
  Synthetic Construct
gtgtaatctggtgagc